(19) United States
(12) Patent Application Publication
(10) Pub. No.: US 2026/0345206 A1
(43) Pub. Date: Nov. 5, 2026

(54) INSTRUMENT SET AND TULIP OF THE SAME

(71) Applicant: TAURUS GMBH & CO. KG, Alzenau (DE)

(72) Inventors: Uwe SIEDLER, Alzenau (DE); Frank HEUER, Alzenau (DE)

(21) Appl. No.: 19/099,333

(22) PCT Filed: May 24, 2023
(86) PCT No.: PCT/EP2023/063961

§ 371 Date: Jan. 28, 2025

(30) Foreign Application Priority Data
Jul. 29, 2022 (DE) 10 2022 002 763.1

(51) Int. Cl. A61B 17/70; A61B 17/00

(52) U.S. Cl. CPC A61B 17/7032; A61B 17/7082; A61B 2017/00526

(57) ABSTRACT

An instrument set, having a tulip extending along its longitudinal axis, a spinal implant having a pedicle screw and serving to couple a connecting rod along a transverse axis of the tulip, and a holding device for temporarily holding the tulip in a holding state of the instrument set during an implant insertion procedure. A transition from the holding state to a release state of the tulip and holding device, and vice versa, involves a rotational movement about an axis, wherein the axis runs with a predominant directional component transverse to the longitudinal axis and parallel to the transverse axis of the tulip.

The present invention relates to the field of spinal implants, in particular such implant systems that include pedicle screws, several of which are interconnected via a connecting rod or a fixing rod to stabilize the spine. In particular, the invention relates to an instrument set comprising a tulip that extends along its longitudinal axis, said tulip forming part of a spinal implant that includes a pedicle screw and serves to connect a connecting rod along a transverse axis of the tulip, and a holding device for temporarily holding the tulip during an implantation process in a holding state of the instrument set, wherein a transition from the holding state to a release state of the tulip and the holding device, and vice versa, involves a rotational movement about an axis.

Implant systems of this kind are, of course, well known to a person skilled in the art; for example, reference is made to US 2012/0041490 A1 or EP 2 376 005B 1. Holding systems of this kind in these and numerous other techniques which rely on straight (purely tangential) grooves in the outer surfaces of the tulip walls, thereby allowing a tangential sliding of a holding instrument but not a rotational screwing of a holding instrument around the longitudinal axis of the tulip, have since been developed, meaning that a holding state between a holding device and the tulip is achieved by rotating the holding instrument about the longitudinal axis of the tulip. This is disclosed, for example, in U.S. Pat. No. 9,050,148 B2 , which describes a groove that follows the cylindrical outer side wall of the tulip circumferentially and is open to the side.

The invention is based on the object of further improving an instrument set of the kind referred to above, in particular against the background of combining ease of operation with a reliable transition from release to the holding state.

This object is achieved by the invention through a development of the instrument set of the kind referred to above, which is substantially characterized in that the axis predominantly runs both transversely to the longitudinal axis and parallel to the transverse axis of the tulip. In spherical coordinates, where the longitudinal axis of the tulip is the z-axis and the transverse axis of the tulip is the x-axis, this therefore corresponds to ϑ>45° and φ<45°. However, these angles are preferably closer to 90° (ϑ) and 0° (φ), preferably not deviating by more than 30° from these values, and more preferably by no more than 20°, in particular by no more than 10°. In a preferred embodiment, the axis of the rotational movement runs substantially parallel to the transverse axis when the tulip and the holding instrument are in the holding state. The axis of the rotational movement is a defined axis defined by the holding instrument and fixed within the system of the holding instrument.

The solution according to the invention eliminates the need for the holding instrument and tulip to be rotatable about the longitudinal axis of the tulip, ensuring a reliable azimuthal alignment between the holding instrument and the tulip during the transition.

Further advantageous embodiments are specified in the dependent claims.

It is therefore preferably provided that during the transition of the instrument set, a shift occurs between an anti-rotational lock that acts radially on the tulip with respect to its longitudinal axis, preventing rotation of the holding device relative to the tulip about its longitudinal axis, and a rotational lock that blocks the rotational movement radially outwards with respect to the longitudinal axis of the tulip. The anti-rotational lock against rotation about the longitudinal axis of the tulip is therefore not only achieved in the final holding state but is already present in an intermediate holding state during the transition from the release state to the holding state. Due to the rotational movement, the forces acting on the tulip during the closing movement are largely balanced in terms of their tangential components, so that effectively radially inwardly directed forces are exerted.

It is further preferred that the transition of the instrument set includes, at least near the holding state, a translational axial movement between the tulip and the holding device along the longitudinal axis of the tulip. This increases the axial depth of the rotational lock.

It is also preferably provided that, in the instrument set, the rotational movement and the axial movement overlap at least temporarily during the transition. To this end, a guide may be provided which is preferably achieved through complementary shapes, such as suitable slopes or curves in the respective coupling areas, for example, on the underside of a radial projection of the instrument and a radially inwardly upwards-facing surface of the tulip.

A tulip for an instrument set according to one of the aforementioned aspects is also provided.

The tulip preferably has at least four coupling areas spaced azimuthally apart from one another on the tulip side for coupling the holding device in the holding state. These are preferably designed to be mutually axis-symmetrical with respect to the transverse axis and, as a preferred variant, achieve the aforementioned force distribution and compensation.

More preferably, a coupling area of the tulip has a notch with a particularly concave base for forming the anti-rotational lock to prevent rotation of the instrument about the longitudinal axis. “Concave” or “concave base shape” in this context does not imply a perfectly mathematical concavity but rather distinguishes it from purely straight (tangential) contours and oppositely curved convex base shapes. For example, if a circle defines a curvature sign around a chord, concavity here means an averaged opposite curvature over the course of the notch base.

The coupling area of the tulip preferably also includes a lower (proximal) axial channel section adjoining the azimuthal notch, which forms the rotational lock region of the rotational lock on the tulip side.

The tulip preferably has multiple upper axial channel sections opening into the upper axial side of the tulip walls, which particularly communicate with the lower channel sections and align with them. While the upper axial channel sections represent an inherently undesirable material weakening of the tulip walls, they are nevertheless preferred for manufacturing reasons.

In one embodiment, the axial channel sections run linearly in cross section when viewed orthogonally to the longitudinal axis and are preferably tangential to the longitudinal axis. However, in another embodiment, these sections could also run polygonally in this cross section. A variant is also preferred in which this a curved trajectory, preferably with the center of the curvature located substantially in the center of the tulip (i.e. at the point where the longitudinal axis intersects the cross-sectional plane). This applies to both the lower and/or upper axial channel sections.

Furthermore, the tulip preferably has side walls of the azimuthal notches pointing towards the distal axial end of the tulip, which include a planar surface section lying in a radial plane or entirely located within a radial plane, in particular at the same axial height. Preferably, one side wall of the tulip has at least two azimuthal notches of this kind.

Even more preferably, the tulip has a side wall of the azimuthal notch pointing towards the proximal axial end of the tulip that is formed as a ramp. The ramped design facilitates the transition between rotational and translational movement and provides the necessary clearance for introducing the component of the rotational lock on the holding instrument side. In one embodiment, mutually facing planar surface sections in the respective base of two azimuthal notches arranged in the same side wall are at an angle to one another that is different from zero, preferably an angle of at least 20 degrees.

In a further preferred embodiment, the tulip has a web area formed azimuthally between two azimuthal notches located on one side wall of the tulip, in particular with an azimuthal dimension decreasing radially outwards. The proportion of the axial channel sections, as viewed in the circumferential direction, is preferably no more than 70% of the azimuthal area of the side wall, preferably no more than 65%, and in particular no more than 60%. The azimuthal dimension of the web area at the web base, i.e. at the radial height of the notch bases and the axial height of the proximal notch side surface, is preferably at least 12°, more preferably at least 18°, and in particular at least 24°.

Two azimuthal notches arranged on different side walls can each have a planar surface section running parallel to one another. Preferably, an azimuthal notch on one side wall is arranged diametrically opposite an azimuthal notch on another side wall.

Preferably, the axial length of an axial end section of the tulip arranged distally to the azimuthal notches and/or that of the axial channels does not exceed 1.6 times the axial dimension of the notch base at its radially innermost point, more preferably not 1.3 times that dimension, and in particular not 1.1 times that dimension. This creates an axially compact design of the tulip. In relation to the latter two values, this ratio preferably also applies to the axial dimension of the notch at its radially outermost point.

A method for manufacturing a tulip is also provided, in which the lower axial channel section is formed after the upper channel section, and the lower channel section is formed in particular before the azimuthal notch. This sequence reduces the risk of damage to the often delicate side walls of the tulip. The holding instrument preferably has two legs that can pivot by rotation about the axis of the rotational movement, with coupling areas of the holding instrument formed on the radial inner side of each leg, matching the number of coupling areas on the tulip. These coupling areas are preferably formed by a radial projection, onto which an axial projection is flanged. The radial projection engages with the azimuthal notch of the tulip, while the axial projection engages with the lower axial channel.

The legs of the holding instrument are preferably biased to achieve a release position, for example by an elastic device. Furthermore, the holding instrument preferably has a locking mechanism that allows it to be locked, against the bias, into a closed state corresponding to the holding state of the instrument. It is evident that this locking mechanism also forms a (primary) rotational lock to prevent a relative pivoting of the legs away from one another. However, based on the findings of the present invention, this rotational lock alone may not always be sufficient in certain cases, meaning that the additional rotational lock provided by the coupling areas of the holding instrument and the tulip offers extra security against an opening relative movement of the legs of this kind.

Further details, features, and advantages of the invention will be apparent from the following description with reference to the accompanying figures, in which:

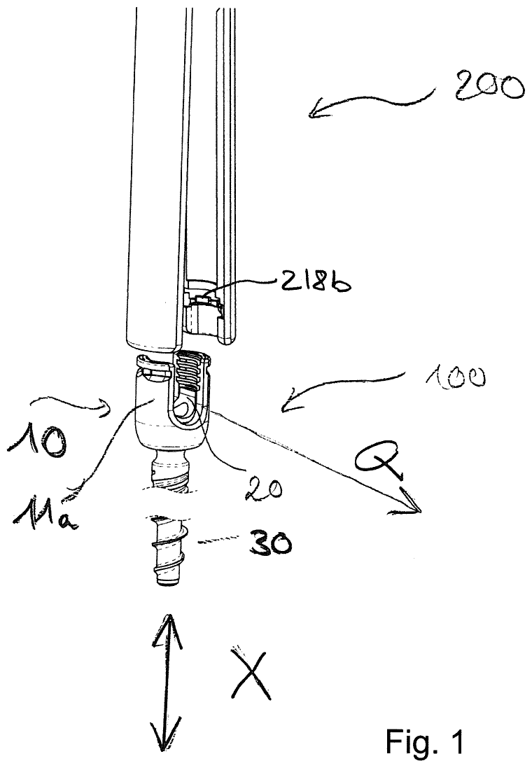

FIG. 1 shows an instrument set with a tulip of a spinal implant and a lower part of a holding instrument,

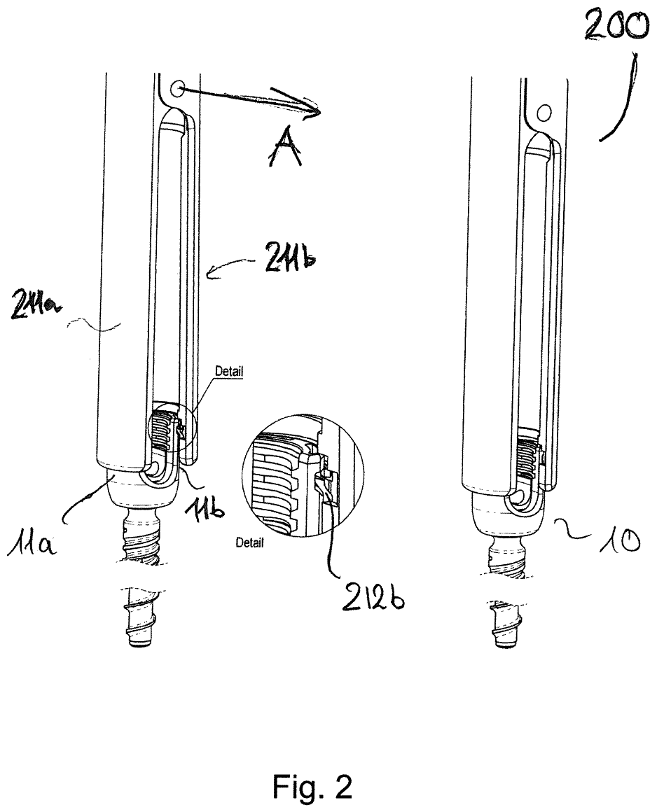

FIG. 2 shows the instrument set from FIG. 1 on the left in an intermediate position to reach the holding state, along with a detailed view, and on the right in the holding state,

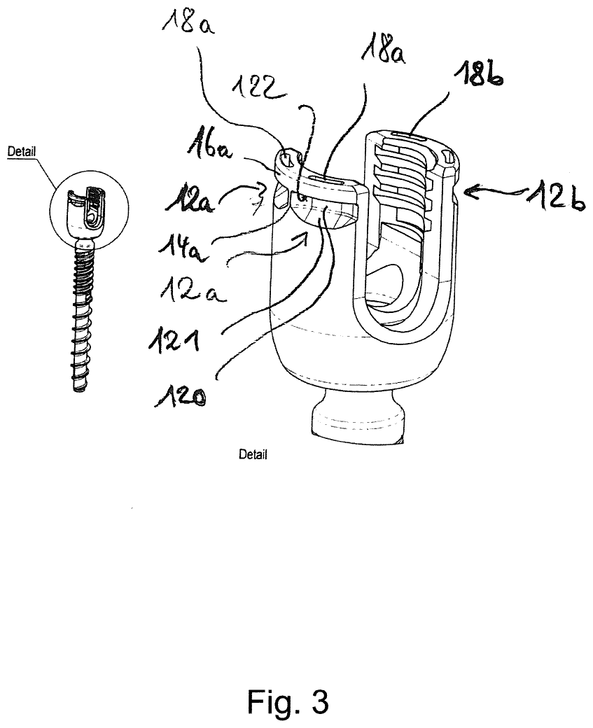

FIG. 3 shows the spinal implant from FIG. 1 along with an enlarged partial view of the upper section with the tulip,

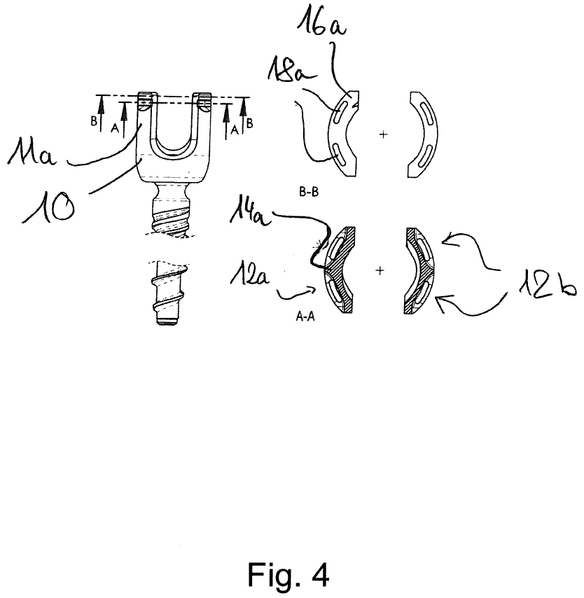

FIG. 4 shows a side view of the spinal implant along with two radial cross-sectional views at different axial heights,

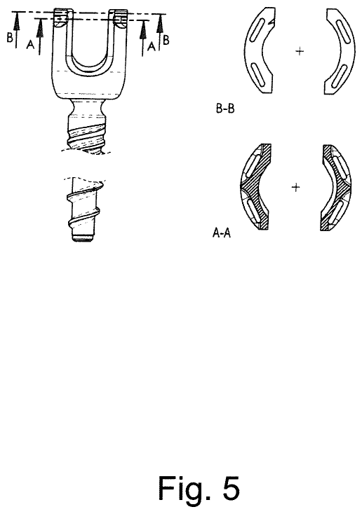

FIG. 5 shows a similar view to FIG. 4 of another embodiment,

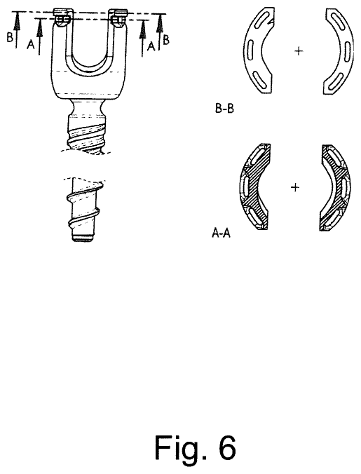

FIG. 6 shows a similar view to FIG. 4 of yet another embodiment,

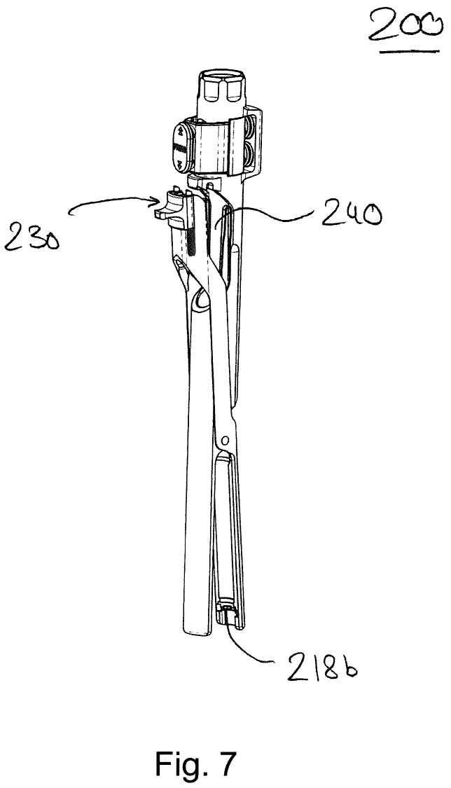

FIG. 7 shows the entire holding instrument from FIG. 1 in a perspective view, in an open state, and

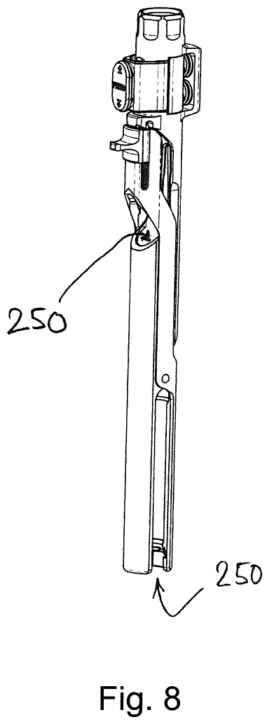

FIG. 8 shows this holding instrument in a closed state corresponding to the holding state depicted on the right in FIG. 2.

The basic functionality of the spinal implant 100 shown in FIG. 1 will be well known to a person skilled in the art. It comprises a bone anchor in the form of a pedicle screw 30 which is mounted in the head of the implant 100, commonly referred to as the tulip 10, in a pivoting manner, particularly in a polyaxially pivoting manner, relative to the longitudinal axis X of the tulip 10. The fundamental structure of the tulip 10 is also typical and well understood by a person skilled in the art. In addition to a receiving area in the lower section for accommodating the (concealed) head of the pedicle screw 30, the tulip has an upper, cylindrical peripheral area from the basic outer shape to form a receiving groove running in the transverse direction Q for accommodating a connecting rod (not shown) that can be used to couple one pedicle screw to another.

In the upper section, two side walls 11a, 11b of the tulip 10 therefore remain, with threads on their inner sides for receiving a set screw (also not shown), such as a grub screw, with which the connecting rod can be fixed relative to the tulip 10. Tightening the set screw also fixes the position of the pedicle screw 30 relative to the tulip 10, not by direct force transmission from the connecting rod onto the screw head, but rather through indirect force transmission by means of an intermediate component commonly referred to as a saddle 20. The saddle 20 at least partially accommodates the screw head in its lower section and is shaped on its upper side to fit the connecting rod running along the transverse direction Q.

It is understood that the invention is not limited in terms of the numerous design possibilities of the basic functionality described thus far, such as the specific designs of the saddle 20, the internal structure of the tulip 10, monoaxiality or polyaxiality, symmetry or asymmetry of the angular ranges available for pivoting relative to the longitudinal axis (axial axis) X, the material selection for the implant, the thread design of the pedicle screw 30, and/or any additional functionalities.

To hold the tulip 10 during the implantation of the spinal implant 100, in particular in minimally invasive procedures, a holding instrument 200 is provided. As is more clearly visible in the holding state shown on the right in FIG. 2 between the instrument 200 and the tulip 10, the holding instrument 200 has two legs, 211a and 211b, which are each coupled to one of the side walls, 11a and 11b, respectively. Although the transition from a release state to this holding state involves a rotational movement about an axis, this axis does not correspond to the traditional rotational axis, which in the prior art is aligned with the longitudinal axis X of the tulip. Instead, the axis A of the rotational movement runs in the transverse direction Q. The instrument is therefore not rotated about the axial axis X relative to the tulip; instead, the coupling movement involves a pivoting motion of the legs 211a and 211b towards each other, induced by the rotational movement, with a corresponding approach to the tulip 10 in a predominantly radial direction relative to the axial axis X and orthogonal to the axis A of the rotational movement which is fixed in the coordinate system of the instrument.

The tulip-side coupling regions 12a and 12b on the side walls 11a and 11b are designed identically on both sides in this exemplary embodiment, meaning that the following description focuses on one side only. As can be seen most clearly in FIG. 3, near its upper axial end, the tulip 10 has concavely shaped cuts 12a that serve as coupling regions. These cuts do not follow the circumferential direction of the convex, substantially cylindrical side wall 11a, but instead act as a rotational lock for the instrument-side counter-coupling parts, preventing the instrument 200 from rotating relative to the tulip 10 about its longitudinal axis X. The counterparts on the instrument are thereby prevented, even in an intermediate holding state before reaching the final holding state, from entering or leaving the concave, notch-like coupling regions 12a due to rotational force. This is best seen in the radial cross-sectional view A-A in FIG. 4.

The concave, notch-like coupling region 12a, hereinafter referred to simply as the notch 12a, comprises, in terminology relating to the radial direction of the axial axis X, a base surface 120, a first side surface 121, and a second side surface 122. The first side surface 121 follows the course of the base surface 120 on its adjoining side and transitions to the convex side wall 11a at its opposite edge. However, it does not lie in the radial plane, but its normal vector, which does not necessarily remain constant along the course of the first side surface 121, exhibits a directional component pointing radially outwards, especially near the base surface 120.

In contrast, the second side surface 122 in this exemplary embodiment is formed in a substantially planar manner and therefore extends in a radial plane with respect to the axial axis X. The axial distance between the second side surface 122 and the first side surface 121 is therefore in any event greater at the azimuthal midpoint transition to the convex outer surface of the side wall 11a than on the base side. In the embodiment shown in FIGS. 1 to 4, it is especially provided that this axial distance increases from radially inwards to radially outwards and also azimuthally from the outer edges towards the azimuthal midpoint of the notch 12a. The notches 12a are spaced apart azimuthally with their azimuthal midpoints separated by approximately 60° in this exemplary embodiment.

The two notches 12a in this exemplary embodiment are separated by a ridge 14a. Due to the concave shape of the base surface 120 of the notches 12a, the ridge 14a has a substantially trapezoidal, almost triangular shape in the radial cross section, so with a significantly larger azimuthal dimension radially inwards compared with radially outward which is 30° in this exemplary embodiment. This shape is specifically intended to provide greater material stiffness to compensate for material weakening in the tulip wall 11a within the region in the axial direction X between the notches 12a and the upper axial end of the tulip 10, as can be seen most clearly in FIG. 3 and, in particular, in the radial cross section B-B in FIG. 4.

Hence, the tulip 10 in this region 16a has two bores 18a running in the axial direction X, which are configured as slots in the azimuthal direction. Their azimuthal extent covers only a portion, in this embodiment approximately half the azimuthal extent of the notches 12a. The inner wall of the slot in the radial direction aligns axially, substantially with the base surface 120 of the notch 12a. In the slot 18a, an axial projection 218a is accommodated in the holding state, which is not visible in FIG. 1 or FIG. 2 due to being obscured by the instrument leg 211a. However, the axial projection 218b mounted on the radial projection 212b, as seen in FIG. 1, is visible in FIG. 7 and is designed to fit into the slot 18b in the opposite side wall 11b. The radially outer boundary of the slot 18a (18b) therefore forms a rotational lock in the holding state, preventing the leg 211a from pivoting about the axis A of the rotational movement of the instrument 200.

The slots 18a, 18b are closed on both azimuthal sides. In this embodiment, the ratio of the material area of the tulip that is not occupied by the slots 18a, 18b and therefore remains part of the tulip, to the total azimuthal extent of the side wall 11a of the tulip in the radial region of the slots 18a, 18b is approximately 50%.

In the embodiment shown in FIG. 5, the slots 18a, 18b are not concentric as in the embodiment in FIG. 4 but are instead tangential, meaning that the slots are straight in design. In the embodiment depicted in FIG. 6, three slots per side wall 11a, 11b are provided, in this case with a concentric arrangement similar to the embodiment in FIG. 4. However, these slots can also be designed straight (tangential), as in the embodiment in FIG. 5, in another embodiment that is not depicted.

As illustrated in FIG. 2, the holding state shown on the right side in FIG. 2 is achieved in that the legs 211a, 211b, by executing the pivoting motion about the axis A, bring the radial projections 212a and 212b, protruding radially inwards from the inner sides of the legs 211a and 211b, predominantly radially into the notches 12a and 12b facing them. The final holding state is achieved by an axial translational movement between instrument 200 and tulip 10, during which the rotational movement about the axis A and this translational movement can already overlap. In the exemplary embodiments shown in the figures, the first side surface 121 and the lower surface of the radial projection 212a, b facing this side surface 121 form a guide that supports and, in particular, can also generate where required a translational axial movement of this kind out of the rotational movement. Correspondingly, when releasing instrument 200 and tulip 10, a translational axial movement along the longitudinal axis X of the tulip can be initiated, and the rotational movement can be guided or initiated by this guide.

In FIGS. 7 and 8, the holding instrument 200 is shown again in its entirety. In the open state depicted in FIG. 7, the legs 211a and 211b are pivoted relative to each other out of their parallel base configuration (FIG. 8) in the holding state. A sliding switch 230, positioned on an extension of leg 211b beyond axis A, can maintain the holding state (FIG. 8) against the restoring force of a spring mechanism 240. However, in the holding state of the tulip, the locking mechanism of the switch 230 is no longer stressed by forces acting on the free ends of the legs 211a and 211b, particularly those directed radially outwards, as these forces are absorbed by the rotational lock formed by the coupling areas of the tulip 10.

The inner channel 250 formed between the legs 211a and 211b extends to the distal end, allowing an internally guided stamp (not shown) to be guided within the instrument to press the connecting rod (also called a fixation rod) explained above into the tulip. The free space in the transverse direction between the legs 211a and 211b of instrument 200 is also of sufficient dimensions for this purpose. This internally guided stamp can also be made hollow inside to permit the axial introduction of an additional instrument part therein, in which case a set screw (not depicted) can be screwed into the thread on the inner side of the tulip walls 11a and 11b. Likewise, a screwing instrument 200 (not depicted) could be guided within the instrument 200 to insert the pedicle screw 30 into a vertebra while the tulip 10 is held by the instrument. In the case of configurations with a saddle 20, said saddle includes an access opening that provides access to the screw head of the pedicle screw 30 for this purpose.

It is understood that the holding instrument can also perform additional functions familiar to a person skilled in the art in connection with the insertion of pedicle screws and holding of the tulips in spinal implants of this kind. To this extent, details of the exemplary embodiments explained above are not to be considered limiting for the invention. Instead, the features of the above description and also the following claims may be essential, individually or in combination, for realizing the invention in its various embodiments.

1-13. (canceled)

14. An instrument set, comprising: a tulip that extends along a longitudinal axis, said tulip forming part of a spinal implant that includes a pedicle screw and serves to connect a connecting rod along a transverse axis of the tulip; and, a holding device configured to temporarily hold the tulip during an implantation process in a holding state of the instrument set, wherein a transition from the holding state to a release state of the tulip and the holding device, and vice versa, involves a rotational movement about an axis, wherein the axis predominantly runs transversely to the longitudinal axis and parallel to the transverse axis of the tulip.

15. The instrument set according to claim 14, further comprising an anti-rotational lock that acts radially on the tulip with respect to the longitudinal axis so as to prevent rotation of the holding device relative to the tulip about the longitudinal axis, and a rotational lock that blocks the rotational movement radially outwards with respect to the longitudinal axis of the tulip, wherein a shift occurs between the anti-rotational lock and the rotational lock during the transition.

16. The instrument set according to claim 14, wherein the transition, at least near the holding state, includes a translational axial movement between the tulip and the holding device along the longitudinal axis of the tulip.

17. The instrument set according to claim 16, wherein the rotational movement and the axial movement overlap at least temporarily during the transition.

18. A tulip for an instrument set according to claim 14.

19. The tulip according to claim 18, comprising at least four coupling areas spaced azimuthally apart from one another on a tulip side for coupling the holding device in the holding state.

20. The tulip according to claim 19, wherein one of the coupling areas has a notch with a concave base so as to form an anti-rotational lock to prevent rotation of the holding device about the longitudinal axis.

21. The tulip according to claim 19, wherein one of the coupling areas includes a lower axial channel section adjoining an azimuthal notch, which forms a rotational lock region of a rotational lock on the tulip side.

22. The tulip according to claim 21, comprising multiple upper axial channel sections that open into an upper distal axial side of the side walls of the tulip.

23. The tulip according to claim 22, wherein the upper axial channel sections communicate with the lower channel sections.

24. The tulip according to claim 23, wherein the upper axial channel sections align with the lower channel sections.

25. The tulip according to claim 21, wherein side walls of the azimuthal notches that point towards a distal axial end of the tulip lie substantially within a radial plane.

26. The tulip according to claim 25, wherein the side walls of the azimuthal notches that point towards the distal axial end of the tulip lie substantially within a radial plane at a same axial height.

27. The tulip according to claim 21, wherein a side wall of the azimuthal notch pointing towards a proximal axial end of the tulip is formed as a ramp.

28. The tulip according to claim 19, comprising a ridge area formed azimuthally between two azimuthal notches formed on one side wall of the tulip.

29. The tulip according to claim 28, wherein the ridge area has an azimuthal dimension decreasing radially outwards.

30. A method for manufacturing a tulip according to claim 18, the method comprising the steps of: forming an upper channel; forming a lower axial channel section after the upper channel section is formed, wherein the lower channel section is formed before an azimuthal notch is formed and before side walls of the tulip are formed by removing a material area located between the side walls.
